(19) United States
(12) Patent Application Publication
(10) Pub. No.: US 2014/0261453 A1
(43) Pub. Date: Sep. 18, 2014

(54) NONCONTACT ENCODER FOR MEASURING CATHETER INSERTION

(71) Applicant: Hansen Medical, Inc., Mountain View, CA (US)

(72) Inventor: Christopher R. Carlson, Menlo Park, CA (US)

(73) Assignee: Hansen Medical, Inc., Mountain View, CA (US)

(21) Appl. No.: 13/795,133

(22) Filed: Mar. 12, 2013

(51) Int. Cl. A61B 19/00; A61B 19/08; A61M 25/09

(52) U.S. Cl. CPC A61B 19/2203; A61B 19/46; A61M 25/09041; A61B 19/081
USPC 128/849; 606/130

(57) ABSTRACT

A robotically controlled surgical system includes a guidewire coupled to a catheter, an active drive system coupled to the guidewire and configured to drive the guidewire in an axial direction; a sensor positioned proximate the guidewire and configured to detect optical characteristics of a surface of the guidewire, and a computer coupled to the sensor. The computer programmed to drive the guidewire in the axial direction a desired distance, detect a first pattern on the surface of the guidewire when the guidewire is at a first axial position, detect a second pattern on the surface of the guidewire when the guidewire is at a second axial position, calculate an actual distance that the guidewire has actually traveled based on the detected first and second patterns, and compare the desired distance to the actual distance.

### BACKGROUND

Robotic interventional systems and devices are well suited for performing minimally invasive medical procedures as opposed to conventional techniques wherein the patient's body cavity is open to permit the surgeon's hands access to internal organs. Advances in technology have led to significant changes in the field of medical surgery such that less invasive surgical procedures, in particular, minimally invasive surgery (MIS), are increasingly popular.

A MIS is generally defined as a procedure that is performed by entering the body through the skin, a body cavity, or an anatomical opening utilizing small incisions rather than large, open incisions in the body. With MIS, it is possible to achieve less operative trauma for the patient, reduced hospitalization time, less pain and scarring, reduced incidence of complications related to surgical trauma, lower costs, and a speedier recovery.

MIS apparatus and techniques have advanced to the point where an elongated catheter instrument is controllable by selectively operating tensioning control elements within the catheter instrument. In one example, four opposing directional control elements wind their way to the distal end of the catheter which, when selectively placed in and out of tension, cause the distal end to steerably maneuver within the patient. Control motors are coupled to each of the directional control elements so that they may be individually controlled and the steering effectuated via the operation of the motors in unison.

However, because the catheter is maneuvered by control motors, a computer, and the like, the surgeon lacks tactile feedback to get an intuitive sense of the location of the distal end of the catheter. Forces driving the catheter may be quantified (e.g., by measuring motor input power) and shown to the surgeon, but the forces themselves are not always indicative of the motion of the catheter that is occurring within the patient. For instance, a slip condition may exist where the catheter is fed into the patient, but the distal end may not be proceeding within the patient commensurate with the motion of the drive motors. That is, advancement of the distal end may stall within the patient while the motors continue to drive the catheter forward. The difference between the drive motion and the actual motion of the distal end defines the amount of slip. Lacking tactile feel for the process, the surgeon is at a disadvantage for not having real-time feedback of the actual location of the distal end. When stalled within the patient, the forces on the catheter are therefore also not proportional to the forces experienced by the motors or drive mechanism that is driving the catheter.

As such, there is a need to measure the slip in a distal end of a catheter and feed that to the surgeon in real-time during, for instance, a surgical operation.

### SUMMARY

A robotically controlled surgical system includes a guidewire coupled to a catheter, an active drive system coupled to the guidewire and configured to drive the guidewire in an axial direction; a sensor positioned proximate to the guidewire and configured to detect characteristics of a surface of the guidewire, and a controller coupled to the sensor. The controller is configured to drive the guidewire in the axial direction a desired distance, detect a first pattern on the surface of the guidewire when the guidewire is at a first axial position, detect a second pattern on the surface of the guidewire when the guidewire is at a second axial position, calculate an actual distance that the guidewire has actually traveled based on the detected first and second patterns, and compare the desired distance to the actual distance.

A method of controlling a guide catheter in a surgical system includes driving a guide catheter in an axial direction and over a desired distance, wherein the guide catheter is coupled to the sheath catheter, detecting a first pattern on a surface of the guide catheter when the guide catheter is at a first axial location, detecting a second pattern on a surface of the guide catheter when the guide catheter is at a second axial location, calculating an actual distance through which the guide catheter traveled based on the first and second patterns, and comparing the desired distance to the actual distance.

A computer readable storage medium having stored thereon a computer program comprising instructions, which, when executed by a computer, cause the computer to drive a guidewire in an axial direction a desired distance, detect a first pattern on a surface of the guidewire when the guidewire is at a first axial position, detect a second pattern on the surface of the guidewire when the guidewire is at a second axial position, calculate an actual distance that the guidewire has actually traveled based on the detected first and second patterns, and compare the desired distance to the actual distance.

### BRIEF DESCRIPTION

FIG. 1 is an illustration of a robotically controlled surgical system, according to one exemplary illustration;

FIG. 2 is an illustration of an exemplary catheter assembly of the surgical system of FIG. 1;

FIGS. 3 and 4 are illustrations of components of the catheter assembly of FIG. 2;

FIG. 5 illustrates a distal end of an exemplary catheter that is controllable by internal control elements;

FIG. 6 illustrate an alternative catheter assembly showing a sensor for detecting a surface of a guide catheter or guidewire;

FIG. 7 a process flow diagram for an exemplary method for determining an amount of movement of a guide catheter or guidewire; and

FIGS. 8A-8C illustrate textured surfaces and patterns detectable using an eigenvalue decomposition.

### DETAILED DESCRIPTION

Referring to FIG. 1, a robotically controlled surgical system 100 is illustrated in which an apparatus, a system, and/or method may be implemented according to various exemplary illustrations. System 100 may include a robotic catheter assembly 102 having a robotic or first or outer steerable complement, otherwise referred to as a sheath instrument 104 (generally referred to as “sheath” or “sheath instrument”) and/or a second or inner steerable component, otherwise referred to as a robotic catheter or guide or catheter instrument 106 (generally referred to as “catheter” or “catheter instrument”). Catheter assembly 102 is controllable using a robotic instrument driver 108 (generally referred to as “instrument driver”). During use, a patient is positioned on an operating table or surgical bed 110 (generally referred to as “operating table”) to which robotic instrument driver 108 is coupled or mounted. In the illustrated example, system 100 includes an operator workstation 112, an electronics rack 114 and associated bedside electronics box (not shown), a setup joint mounting brace 116, and instrument driver 108. A surgeon is seated at operator workstation 112 and can monitor the surgical procedure, patient vitals, and control one or more catheter devices.

System components may be coupled together via a plurality of cables or other suitable connectors 118 to provide for data communication, or one or more components may be equipped with wireless communication components to reduce or eliminate cables 118. Communication between components may also be implemented over a network or over the internet. In this manner, a surgeon or other operator may control a surgical instrument while being located away from or remotely from radiation sources, thereby decreasing radiation exposure. Because of the option for wireless or networked operation, the surgeon may even be located remotely from the patient in a different room or building.

Referring now to FIG. 2, an instrument assembly 200 includes sheath instrument 104 and the associated guide or catheter instrument 106 mounted to mounting plates 202, 204 on a top portion of instrument driver 108. During use, catheter instrument 106 is inserted within a central lumen of sheath instrument 104 such that instruments 104, 106 are arranged in a coaxial manner. Although instruments 104, 106 are arranged coaxially, movement of each instrument 104, 106 can be controlled and manipulated independently. For this purpose, motors within instrument driver 108 are controlled such that carriages coupled to mounting plates 204, 206 are driven forwards and backwards on bearings. As a result, a catheter coupled to guide catheter instrument 106 and sheath instrument 104 can be controllably manipulated while inserted into the patient, as will be further illustrated. Additional instrument driver 108 motors may be activated to control bending of the catheter as well as the orientation of the distal tips thereof, including tools mounted at the distal tip. Sheath catheter instrument 106 is configured to move forward and backward for effecting an axial motion of the catheter, e.g., to insert and withdraw the catheter from a patient, respectively.

Referring to FIG. 3, an assembly 300 includes sheath instrument 104 and guide or catheter instrument 106 positioned over their respective mounting plates 206, 204. In the illustrated example, a guide catheter instrument member 302 is coaxially interfaced with a sheath catheter member 304 by inserting the guide catheter instrument member 302 into a working lumen of sheath catheter member 304. Sheath catheter member 304 includes a distal end that is manipulable via assembly 300, as will be further discussed in FIG. 5. Sheath instrument 104 and guide or catheter instrument 106 are coaxially disposed for mounting onto instrument driver 108. However, it is contemplated that a sheath instrument 108 is used without guide or catheter instrument 106, or guide or catheter instrument 106 is used without sheath instrument 104 and may be mounted onto instrument driver 108 individually.

When a catheter is prepared for use with an instrument, its splayer is mounted onto its appropriate interface plate. In this case, sheath splayer 308 is placed onto sheath interface plate 206 and a guide splayer 306 is placed onto guide interface plate 204. In the illustrated example, each interface plate 204, 206 has respectively four openings 310, 312 that are designed to receive corresponding drive shafts 314, 316 (FIG. 4 illustrates an underside perspective view of shafts 314, 316) attached to and extending from the pulley assemblies of the splayers 308, 306).

Operator workstation 112 may include a computer monitor to display a three dimensional object, such as a catheter instrument 502 as illustrated in FIG. 5. Catheter instrument 502 may be displayed within or relative to a three dimensional space, such as a body cavity or organ, e.g., a chamber of a patient's heart. In one example, an operator uses a computer mouse to move a control point around the display to control the position of catheter instrument 502.

Turning now to FIGS. 3 and 4, an exemplary sheath instrument 104 and catheter instrument 106 are described in further detail. According to one exemplary illustration, sheath instrument 104 may include a sheath splayer 308 having drive shafts 314. Catheter instrument 106 may include a guide splayer 306 having drive shafts 316. Drive shafts 316 are each coupled to a respective motor within instrument driver 108 (motors not shown). When 4-wire catheter 304 is coupled to instrument driver 108, each drive shaft 316 thereof is thereby coupled to a respective wire 504-510 (see FIG. 5). As such, a distal end 512 of catheter 304 can be articulated and steered by selectively tightening and loosening wires 504-510. Typically, the amount of loosening and tightening is slight, relative to the overall length of catheter 304. That is, each wire 504-510 typically need not be tightened or loosened more than perhaps a few centimeters. As such, the motors that tighten/loosen each wire typically do not rotate more than, for example, ¾ of a rotation.

Splayer 314 and drive shaft 316 have pin/screw combinations and flats. These features act as a key and match with corresponding features in the output shafts of the robotic system. The robotic system presents its output shaft in a fixed orientation upon boot up to receive the keyed pins of the splayer. A typical motor and gear box in a robotic system includes a hard stop in a gear box that allows the motor to find a home point every time the system is booted up. The encoder can then index from this point and position the keyed output shafts at any desired location. It is beneficial for the output shafts of the robotic system to rotate less than one full revolution, which enables a hard stop to be designed into the rotation mechanism.

Referring to FIG. 6, a robotic instrument assembly 600 is illustrated that is an alternative to instrument assembly 200. Assembly 600 includes an instrument driver 602. A sterile drape 604 is positioned over instrument driver 602 and isolates non-sterile components from sterile components. Incidentally, although not illustrated in FIG. 2, a sterile drape may also be included in instrument assembly 200 and surrounding instrument driver 108 (which is non-sterile) from sterile components such as sheath and catheter instruments 104, 106, catheter 304, etc. Instrument assembly 600 includes an active drive system 606 that is coupled to a guide catheter or guidewire 608, which passes through catheter splayer 610. Catheter 304 extends therefrom and is, in one embodiment, a sheath catheter. Active drive 606 according to one embodiment, and in lieu of or in addition to catheter instruments 106, is used to axially and/or rotationally move catheter or guidewire 608 and allows for continuous feed of catheter or guidewire 608.

A sensor 612 is positioned within instrument driver 602 and an optically clear section 614 is positioned within sterile drape 604. Sensor 612 may be based on CMOS technology or may be based on CCD technology, as examples. According to one optional embodiment, a lens 616 is positioned between optically clear section 614 and guide catheter or guidewire 608. In another embodiment, however, lens 616 is positioned on the other side of sterile drape 604 and is instead positioned between optically clear section 614 and sensor 612. The sensor 612 may be positioned proximal of the active drive system 606 as shown to detect movement of the wire or catheter as it enters the active drive system 606 or can also be positioned distal of the active drive system 606 (between the active drive 606 and the splayer 610) to detect movement of the guidewire or catheter as it exits the active drive. Guide catheter 608 includes a textured surface 618 which is detectable via sensor 612 as light emitting therefrom passes through optically clear section 614 and optional lens 616. The light emitting is generally reflected from light passing to textured surface 618 that is illuminated from surrounding diffuse light. However, according to one embodiment, a light source 620 may be provided that is directed toward textured surface to provide active illumination thereof. As such, a linear position of guide catheter 608 may be detected using sensor 612, as will be further described.

Thus, whether instrument assembly 200 or instrument assembly 600 is employed, an optically identifiable textured surface such as textured surface 618 may be positioned on guide catheter 608 or guide catheter instrument member 302 (FIG. 3). Textured surface 618 is illuminated passively by surrounding light, or actively by a light source such as light source 620. Light emitting from textured surface 618 passes from a sterile side of surgical system 100 to a nonsterile side through sterile drape 604 and more specifically through optically clear section 614. The light passes through lens 616 in one embodiment and lens may be positioned on either side of sterile drape 604.

Referring to FIG. 7, motion of guide catheter 608 is detected using method or algorithm 700. Starting at step 702, motion of the guide catheter is commanded at step 704. Optical data is detected from the catheter surface at step 706 and at a known time, and decomposed at step 708. Decomposition at step 708 is performed using an eigenvalue decomposition. The eigenvalue decomposition of the optical data is performed at a rate that is significantly faster than the rate at which guide catheter 608 passes. That is, the decomposition is performed in a fraction of the time that it takes for discernible features of a textured pattern to pass proximate to sensor 612. In one embodiment the decomposition is performed in less than 10 ms.

The eigenvalue decomposition may be performed using known methods. According to one method, open source code is available with ready-to-use function(s) that handle visual inputs such as images, video files, or motion data, as examples. The function(s) are incorporated into a workstation, such as workstation 112, and further incorporated into existing programs (e.g., for image processing) or standalone programs as, for instance, an executable file. Once the images are obtained they may be manipulated to identify the features of interest. For instance, a color image may be converted to a grayscale image. Or, subsequent images may be placed into subsequent frames, and features (such as recognizable texture features, or B/W patterns, or B/W overall content, as examples) may be assessed to determine a an location of the feature. In one example a Lucas Kanade algorithm makes an analysis based on assumptions that include pixel brightness, total assumed motion between subsequent frames, and an assumption that pixels that inhabit a small area belong to one another in a larger image, and are moving in a similar direction from image to subsequent image. Once the tracking features or patterns are identified, they are tracked from image to image and local motion is obtained therefrom. The process continues as the features track through the field of view, and new features or patterns are identified for tracking as prior features pass out of the field of view.

The optical data detected from the surface, such as textured surface 618, is analyzed to detect a known pattern or recognizable feature that can be used to track motion of the textured surface. Examples of textured surfaces are illustrated in FIGS. 8A, 8B, and 8C. FIG. 8A shows a textured surface 800 having a textured pattern 802 with distinguishable features 804. Examples of textured pattern 802 include but are not limited to a metal braid or a wire. The eigenvalue decomposition performed at step 708 is thereby conducted and features 804 are recognized during subsequent assessments thereof. That is, at step 706 the optical data is detected from catheter surface 618 and at step 708 the optical data is decomposed using the eigenvalue decomposition. At step 710 the distance moved by guide catheter 608 is determined. That is, image data acquisition and decomposition is performed subsequently at rates that are in excess of the motion of guide catheter 608. In such fashion the distance moved by guide catheter 608 can be determined based on, for instance, the distance moved by one or more of distinguishable features 804. As such, because the time between image acquisitions is known and because the distance moved by distinguishable features 804 is determined in subsequent steps, the velocity of distinguishable features 804 is thereby determined at step 712. In other words, distinguishable features 804 are detected as a first pattern and at a first time, and a second pattern is subsequently obtained that includes some or all of the distinguishable features as they move through the field of view. Distinguishable features are continually updated through, for instance, pattern recognition according to one embodiment.

In addition, because the commanded (or intended) motion of guide catheter 608 is always known, the expected displacement and velocity of the guidewire or guide catheter can be compared to the actual displacement and velocity detected by the sensor 616, the amount of slip of guide catheter 608 can likewise be determined or calculated at step 714. That is, an amount of slip is determined as a difference between the intended axial motion of guide catheter 608 and the actual motion that is observed by the sensor. Using the position and/or velocity information the commanded position and/or velocity measurement(s) can be compared to the actual respective position and/or velocity. The difference therebetween, generally described as slip, can be used to notify the user when the device is tracking well or not or could stop the motion automatically.

For viscoelastic materials, the amount of slip in the system is proportional to the force on the catheter or guidewire. Thus, the slip data can also be used to predict insertion force. The insertion force is calculated based on the calculated velocity or slip and the known stiffness of the catheter. As one example, based on the velocity determined at step 712, an amount of insertion force of guide catheter 608 can be determined as:

F=C*(V_{command}−V_{actual})/V_{command} Eqn. 1.

The term V_{command} refers to the commanded velocity of guide catheter 302 or 608, and V_{actual} refers to the actual or measured velocity that is obtained via the optical measurements described. C is a constant based on the stiffness of the guide catheter 302 or 608. The relationship between slip and force can be calibrated for guide catheter 302 or 608, as examples.

Thus, referring back to FIG. 7, at step 716 the force on guide catheters 302 or 608 can be obtained based on earlier obtained calibration data. At step 718, method or algorithm 700 thereby determines whether additional motion data is to be obtained and, if so 720, control returns to step 706 where additional optical data may subsequently be obtained. If no additional data is desired 722 (e.g., the end of a surgical process), then the process ends at step 724.

As stated, the velocity of guide catheter 302 or 608 may be optically measured by identifying features such as distinguishable features 804 as illustrated regarding textured surface 800 of FIG. 8A. However, instead of relying on detecting distinguishable features 804, according to other embodiments, guide catheters 302 or 608 can have surfaces otherwise altered or patterned such that the velocity thereof may be determined without having to rely upon identification of particular features 804. For instance, FIG. 8B illustrates a pattern 820 that is observable within a field of view 822. Pattern 820 (illustrated for simplicity as having the same textured pattern as in FIG. 8A, but it is understood that the textured pattern of features 804 is typically continuously different along a length of surface 618) includes a “white” portion 824 and a “dark” portion 826. That is, pattern 820 is a repeating pattern of white and dark patches which may be distinguishable in the acquired image data. As pattern 820 thereby is translated along and passes within a field of view of sensor 612, a ratio of white to dark may be continuously calculated until equal ratios of each are observed. Because the pattern has a known period or repeating pattern between light and dark patches, the velocity V_{actual} can be calculated based on travel between periods of maximum white/dark ratio, from which slip, force, etc. . . . can be obtained.

Similarly, referring to FIG. 8C, a repeating pattern of white 842 and dark 844 portions may be provided that allow pattern recognition to obtain a higher resolution of travel in real-time than, for instance, that shown in FIG. 8B. That is, pattern 820 of FIG. 8B provides accurate position information when the ratio of white to dark is equal, but pattern 840 of FIG. 8C provides a detectable resolution in the white/dark pattern that can translate to a higher rate of slip and force feedback to the surgeon.

The repeating patterns of black and white of FIGS. 8B and 8C may be positioned thereon using any known surface treatment, including but not limited to paint, oxidation, and ink, as examples.

Further, the amount of slip and/or force determined can be displayed to the surgeon via workstation 112, which may be displayed with other detected features as well, to include for instance estimates or measurements related to system vibration, an estimate of viscosity of the material through which the catheter is traveling, and notifications to the surgeon if high forces, slip, vibration, viscosity are encountered during the procedure. Such notifications can be via a pop-up warning, a blinking light on the computer, or an audio signal corresponding to the types of issues that may be encountered, as examples.

Operator workstation 112 may include a computer or a computer readable storage medium implementing the operation of drive and implementing method or algorithm 700. In general, computing systems and/or devices, such as the processor and the user input device, may employ any of a number of computer operating systems, including, but by no means limited to, versions and/or varieties of the Microsoft Windows® operating system, the Unix operating system (e.g., the Solaris® operating system distributed by Oracle Corporation of Redwood Shores, Calif.), the AIX UNIX operating system distributed by International Business Machines of Armonk, N.Y., the Linux operating system, the Mac OS X and iOS operating systems distributed by Apple Inc. of Cupertino, Calif., and the Android operating system developed by the Open Handset Alliance.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many faults, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

1. A robotically controlled surgical system comprising:
a guidewire coupled to a catheter;
an active drive system coupled to the guidewire and configured to drive the guidewire in an axial direction;
a sensor positioned proximate to the guidewire and configured to detect characteristics of a surface of the guidewire; and
a controller configured to:
drive the guidewire in the axial direction a desired distance;
detect a first pattern on the surface of the guidewire when the guidewire is at a first axial position;
detect a second pattern on the surface of the guidewire when the guidewire is at a second axial position;
calculate an actual distance that the guidewire has actually traveled based on the detected first and second patterns; and
compare the desired distance to the actual distance.

2. The surgical system of claim 1, wherein the controller is further configured to determine an actual velocity of the guidewire between the first and second axial positions based on the detected first and second patterns and based on known times, obtained by the computer, when the first and second patterns are detected.

3. The surgical system of claim 1, wherein the controller is further configured to determine an amount of slip based on the comparison between the desired distance and the actual distance.

4. The surgical system of claim 1, wherein the controller is further configured to determine a force applied to the guidewire based on the comparison between the desired distance and the actual distance.

5. The surgical system of claim 1, further comprising:
a sterile drape positioned between the guidewire and the sensor; and
an optically clear section of the sterile drape positioned such that the detected characteristics of the surface are optical and pass to the sensor through the optically clear section.

6. The surgical system of claim 5, further comprising a lens positioned between the sensor and the surface.

7. The surgical system of claim 1, wherein the first pattern and the second pattern include one of discernible features of the surface and a repeating pattern of light and dark areas of the surface.

8. A method of controlling a guide catheter in a surgical system comprising:
driving a guide catheter in an axial direction and over a desired distance, wherein the guide catheter is coupled to the sheath catheter;
detecting a first pattern on a surface of the guide catheter when the guide catheter is at a first axial location;
detecting a second pattern on a surface of the guide catheter when the guide catheter is at a second axial location;
calculating an actual distance through which the guide catheter traveled based on the first and second patterns; and
comparing the desired distance to the actual distance.

9. The method of claim 8, further comprising:
determining a first time when the first pattern is detected;
determining a second time when the second pattern is detected;
determining an actual velocity of the guide catheter between the first and second axial positions based on the detected first and second patterns and based on the first and second times.

10. The method of claim 8, further comprising determining an amount of slip based on the compared desired distance and actual distance.

11. The method of claim 8, further comprising determining a force applied to the guide catheter based on the comparison between the desired distance and the actual distance.

12. The method of claim 8, further comprising:
positioning a sterile drape between the guide catheter and a sensor that is used to detect the first and second patterns; and
positioning a sterile drape having an optically clear section such that the detected first and second patterns pass to the sensor through the optically clear section.

13. The method of claim 12, further comprising positioning a lens between the sensor and the guide catheter such that the first and second patterns are detected through the lens.

14. A computer readable storage medium having stored thereon a computer program comprising instructions, which, when executed by a computer, cause the computer to:
drive a guidewire in an axial direction a desired distance;
detect a first pattern on a surface of the guidewire when the guidewire is at a first axial position;
detect a second pattern on the surface of the guidewire when the guidewire is at a second axial position;
calculate an actual distance that the guidewire has actually traveled based on the detected first and second patterns; and
compare the desired distance to the actual distance.

15. The computer readable storage medium of claim 14, wherein the computer is further caused to:
determine an actual velocity of the guidewire between the first and second axial positions based on the detected first and second patterns and based on known times, obtained by the computer, when the first and second patterns are detected.

16. The computer readable storage medium of claim 14, wherein the computer is further programmed to determine an amount of slip based on the comparison between the desired distance and the actual distance.

17. The computer readable storage medium of claim 14, wherein the computer is further caused to determine a force applied to the guidewire based on the comparison between the desired distance and the actual distance.

18. The computer readable storage medium of claim 14, wherein the computer is further programmed to detect optical characteristics of the surface after having passed through an optically clear section of a sterile drape that is positioned between the guidewire and the sensor.

19. The computer readable storage medium of claim 18, wherein the computer is further programmed to detect the optical characteristics of the surface after having passed through a lens that is positioned between the sensor and the surface.

20. The computer readable storage medium of claim 14, wherein the first pattern and the second pattern include one of discernible features of the surface and a repeating pattern of light and dark areas of the surface.
